(19) United States
(12) Patent Application Publication
(10) Pub. No.: US 2025/0248389 A1
(43) Pub. Date: Aug. 7, 2025

(54) Large-scale stabilized nanoemulsion formulations, methods for their manufacturing, and their uses

(71) Applicant: Duquesne University of the Holy Spirit, PITTSBURGH, PA (US)

(72) Inventors: Jelena M. Janjic, Pittsburgh, PA (US); Vijay S. Gorantla, Winston-Salem, NC (US); Caitlin V. Crelli, Yonkers, NY (US); Anneliese N. Troidle, Branford, CT (US)

(73) Assignee: Duquesne University of the Holy Spirit, PITTSBURGH, PA (US)

(21) Appl. No.: 18/911,943

(22) Filed: Oct. 10, 2024

Related U.S. Application Data
(60) Provisional application No. 63/543,449, filed on Oct. 10, 2023.

(51) Int. Cl. A01N 1/126; G01N 21/359; G01N 33/483

(52) U.S. Cl. CPC A01N 1/126; G01N 21/359; G01N 33/4833

(57) ABSTRACT

The present invention is a formulation for oxygen delivery for organ preservation comprising a scalable stabilized nanoemulsion, and a method for using it. The nanoemulsion embraces a hydrocarbon lipid; a fluorocarbon or perfluorocarbon; water; a nonionic surfactant; and optionally a quaternary ammonium compound, so that droplets of the nanoemulsion have a droplet size of from about 90 nm to about 120 nm and wherein the diameter of the droplets does not change by more than 20% upon storage for at least 12 months.

### CROSS-REFERENCE TO RELATED APPLICATIONS

This application claims priority to U.S. Provisional Patent Application No. 63/543,449, filed Oct. 10, 2023, which is entirely incorporated by reference here.

### STATEMENT REGARDING FEDERALLY SPONSORED RESEARCH OR DEVELOPMENT

This work was supported by the U.S. Air Force Defense Research Sciences Program Award FA8650-20-2-6224 and the Congressionally Directed Medical Research Award HT9425-24-1-0828. The U.S. government has certain rights in the invention.

### BACKGROUND

Fluorocarbons are dense transparent clear oils with a refractive index close to that of water and with specific weight of more than 1.5 g/mL. To stabilize fluorocarbons in aqueous solutions, they must be dispersed as small droplets (such as less than 150 nm) as nanoemulsions, stabilized by surfactants, using either polymer-based materials (e.g., pluronics) or phospholipids. However, due to their high density, fluorocarbon nanoemulsion droplets tend to sediment over time, which challenges their long-term colloidal stability. In addition, preparing large amounts of nanoemulsions tend to result in quick phase separation and overall colloidal and chemical instability. In addition, emulsified fluorocarbon compositions should meet strict requirements in terms of stability and sterility for example in order to be used in applications as oxygen carriers.

### SUMMARY

In the present invention, fluorocarbon nanoemulsions have been stabilized and are scalable and prepared at large-scale. Large-scale or scalable stabilized fluorocarbon nanoemulsion formulations may include fluorocarbons, hydrocarbons, and water.

Methods for the preparation of these large-scale stabilized fluorocarbon nanoemulsion formulations may include the microfluidization of a fluorocarbon phase, a hydrocarbon phase and an aqueous phase.

Methods for the use of these large-scale stabilized fluorocarbon nanoemulsion formulations may include preservation during Vascularized Composite Allotransplantation (VCA). These preservation methods that include limbs, face, penis, uterus, abdominal wall, whole eye, flaps at any location, in humans and animal models, for clinical and preclinical applications, including research applications. Further, the large-scale stabilized fluorocarbon nanoemulsion formulations may be used in machine supported preservation during all types of VCA and solid organ transplantation including kidneys, lungs, heart, and liver.

### BRIEF DESCRIPTION OF DRAWINGS

FIGS. 1A-1E show multiple linear regression output to predict size change after thermal storage and Cmax in oxygen release of large-scale stabilized fluorocarbon nanoemulsion formulations. FIG. 1A shows a contour plot representing the formulation space in X1 and X2 identified by regression models for both responses. Out-of-specification regions for Cmax and Thermal Stability % Size Change are shown on the left and right of FIG. 1A, respectively. FIGS. 1B and 1C show a regression term estimates for Cmax (FIG. 1B) and Thermal Stability % Size Change (FIG. 1C). FIGS. 1D and 1E show observed by predicted graphs visualize the goodness of fit for both models, where Cmax is shown in FIG. 1D and Thermal Stability % Size Change is shown in FIG. 1E.

FIG. 2A shows a large-scale production of perfluorocarbon nanoemulsions (PFC-NEs) (100× scale up) from small scale (24 mL) to large-scale (2.4 L). FIG. 2B shows the size distribution remaining in the same shape and in the same range 90-110 nm upon scale up 100×. FIG. 2C shows a NIRF signal remaining unchanged upon 100× scale up from 24 to 2400 mL of NIRF labeled PFC-NEs. FIG. 2D shows the lack of change in size distribution between batches.

FIGS. 3A-3F illustrate a PFC-NEs quality testing. FIG. 3A shows colloidal stability of and FIG. 3B shows the fluorescence stability of perfluorocarbon nanoemulsions upon storage; FIG. 3C shows the size distribution profiles of perfluorocarbon nanoemulsions and FIG. 3D shows fluorescence sampled from PFC-NEs during machine perfusion of human limbs over 16 h; FIG. 3E shows the size of PFC-NEs sampled across 24 h perfusion; FIG. 3F shows their sterility.

FIGS. 4A-4C shows the NIRF imaging of PFC-NE perfusion through non-human primate limbs in comparison to Cold Static Preservation with Custodiol® (HTK) perfusion. FIG. 4A shows representative images of the perfused NHP limb in grayscale and rainbow, intensity scale; FIG. 4B shows raw fluorescence signal quantified from NIRF images. FIG. 4C shows normalized signal per surface area. The signal is quantified and compared between the limb with PFC-NEs vs HTK perfused limbs.

FIGS. 5A-5D shows the size distribution and fluorescence signal of PFC-NEs throughout perfusion. Samples were taken every 30 minutes during perfusion of PFC-NEs for real-time monitoring of product integrity. FIGS. 5A and 5C show the size distribution of PFC-NEs.

FIG. 6 shows a comparison of oxygen concentration in cannulated limbs. Comparison of measured oxygen concentration in cannulated limbs perfused with a nanoemulsion formulation according the present invention and with 1×PBS. p<0.0001 when comparing oxygenation results of limb perfused with the nanoemulsion formulation and limb perfused with 1×PBS.

FIGS. 7A-7B show NIRF imaging of cannulated limbs. FIG. 7A shows processed Fluobeam images of cannulated rat hind limbs. Fluobeam was positioned at 23 cm above the bench top and all images selected for processing were taken at the 5 ms_x_2 setting.

FIG. 7B shows luantified fluorescence signal of cannulated rat hind limbs perfused with a nanoemulsion formulation according to the invention.

FIG. 8 shows an example of the use of a NIRF labeled nanoemulsion formulation according to the invention in a commercial grade perfusion system.

### DETAILED DESCRIPTION

Large-scale scalable stabilized fluorocarbon nanoemulsion formulations for oxygen delivery for organ preservation comprising a scalable stabilized nanoemulsion may comprise hydrocarbon lipids, fluorocarbons, water, nonionic surfactants, and optional quaternary ammonium compounds. Droplets of the nanoemulsion may have a droplet size of from about 90 nm to about 120 nm and the diameter of the droplets may be such that it does not change by more than 20% upon storage for at least 12 months.

Methods for the use of these large-scale scalable stabilized fluorocarbon nanoemulsion formulations may include limbs, face, penis, uterus, abdominal wall, whole eye, flaps at any location, in humans and animal models, for clinical and preclinical applications, including research applications. Further, the large-scale stabilized fluorocarbon nanoemulsion formulations may be used in machine supported preservation during all types of VCA and solid organ transplantation including kidneys, lungs, heart, liver. Such use is particularly beneficial because the large-scale stabilized fluorocarbon nanoemulsion formulations include a small droplet size (ideally about 100 nm), which allows sterile filterability and reduce sedimentation, a narrow size distribution (e.g., polydispersity index <0.2) and a high fluorine concentration (20-30% w/v) to maximize oxygen carrying capacity while keeping viscosity sufficiently close to normal saline.

In addition, large-scale scalable stabilized fluorocarbon nanoemulsion formulations may be used in applications from imaging to oxygen delivery. The manufacturing process development and quality control for the preparation of these large-scale stabilized fluorocarbon nanoemulsion formulations was adapted for clinical translation. These large-scale stabilized fluorocarbon nanoemulsion formulations replenish oxygen in lieu of whole blood without the risk of infection, may be commercially manufactured on large-scale (>1 L), are chemically and biologically inert, and are stable at elevated temperature (>50° C.) or prolonged storage.

The large-scale scalable stabilized fluorocarbon nanoemulsion formulations were developed for example as clinically viable oxygen carriers and provide the following: 1) colloidal and chemical stability related to shelf-life and applicability in challenging environments (e.g. exposure to changing temperatures, high or low temperatures during storage and use, shipment); 2) fluorocarbons and surfactants maximize oxygen delivery and minimize body retention/accumulation as well as minimize engagement of the immune system. 3) manufacturing process established for production on large-scale and batch to batch quality control throughout the product life-time.

The large-scale scalable stabilized fluorocarbon nanoemulsion formulation provide small-size (<200 nm) oil-in-water emulsion droplets with a surface-area-to-volume ratio that allows for effective loading of multiple payloads (drugs or oxygen), targeted intra-or-extracellular release, and the ability to functionalize the surface with targeting ligands and imaging moieties (cell targeting agents, metal chelates, imaging dyes, etc.).

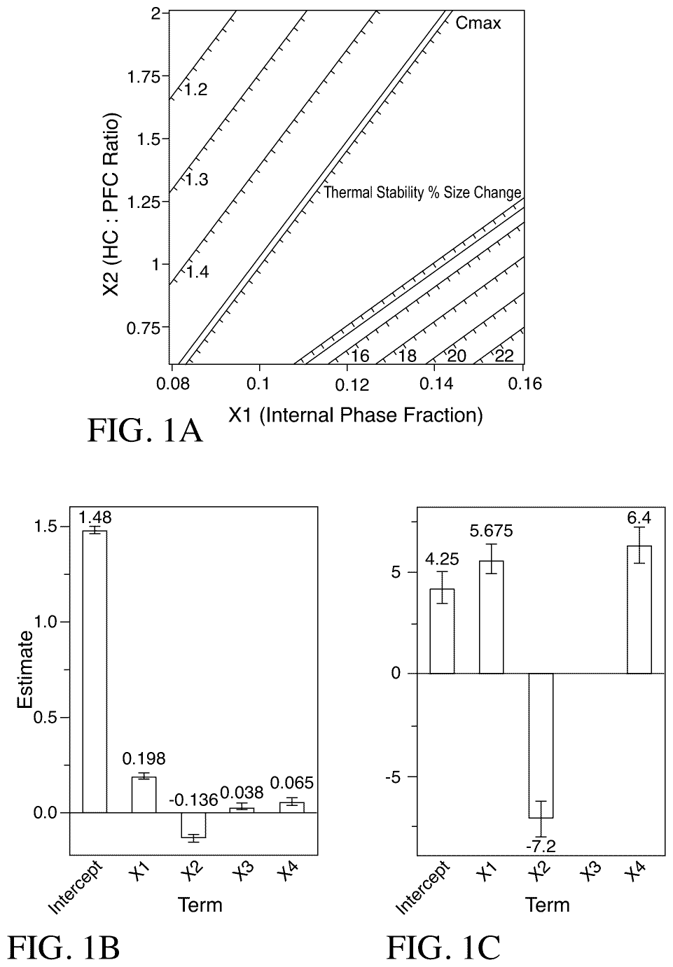

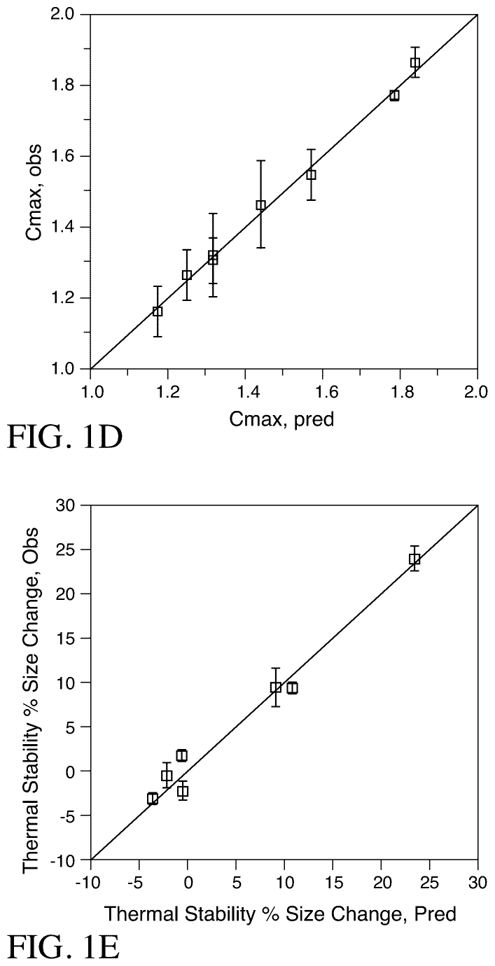

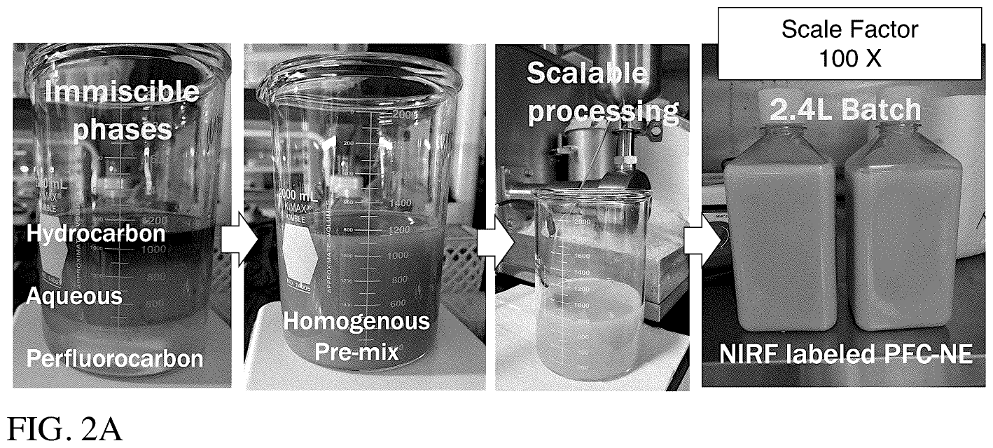

The large-scale scalable stabilized fluorocarbon nanoemulsion formulations may be prepared from three immiscible liquids: PFC (fluorous phase), hydrocarbon (synthetic or natural, organic phase) and water (aqueous) phase implementing Quality by Design (QbD) approaches. Multiple linear regression (MLR) modeling established clear relationships between thermal stability, fluorocarbon content and oxygen release (FIGS. 1A-IE). Processing (microfluidizer processor pressure, number of passes, temperature) and composition parameters (PFC to oil ratio, PFC concentration, aqueous phase pH and salinity) were successfully scaled up in large-scale stabilized fluorocarbon nanoemulsion formulations for oxygen delivery in batch sizes of 600 mL, 1.2 L, and 2.4 L.

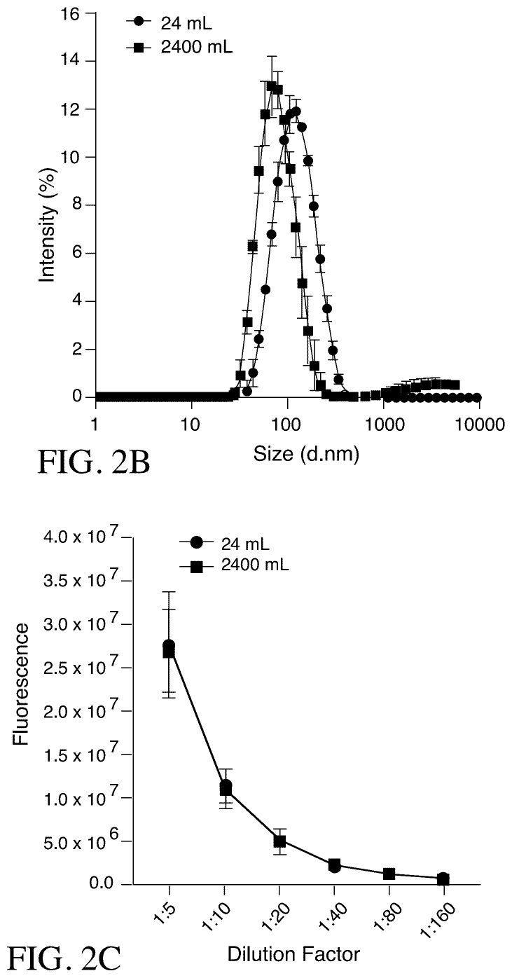

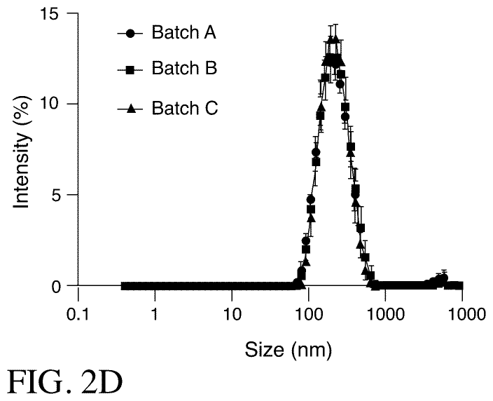

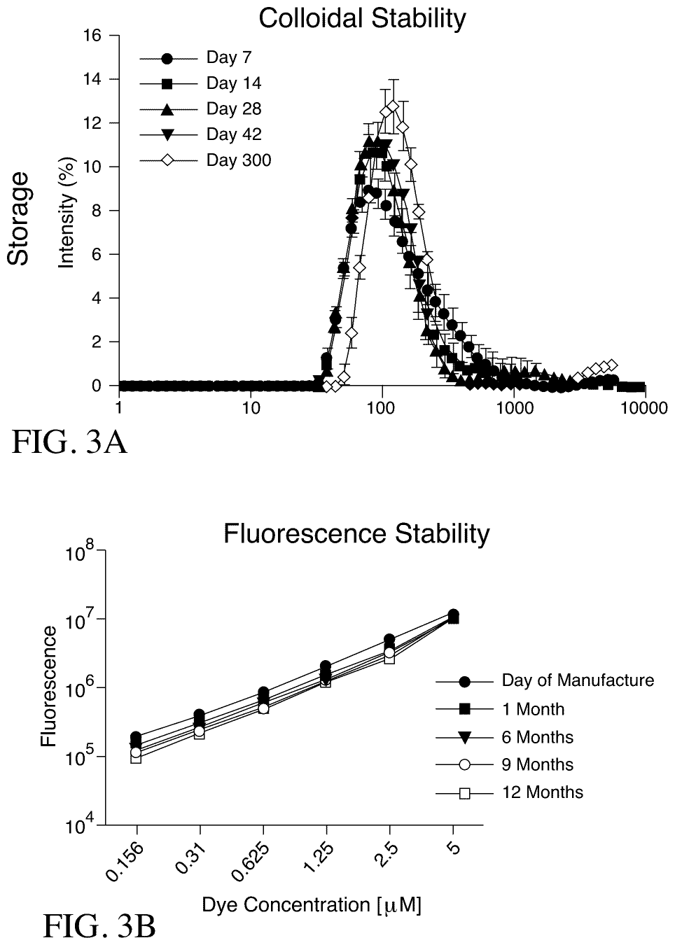

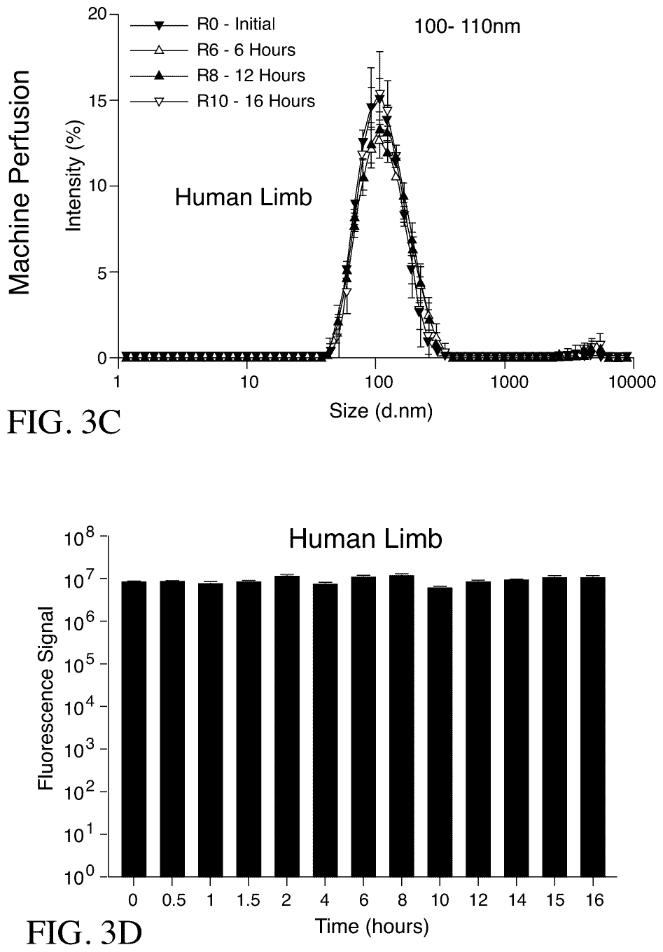

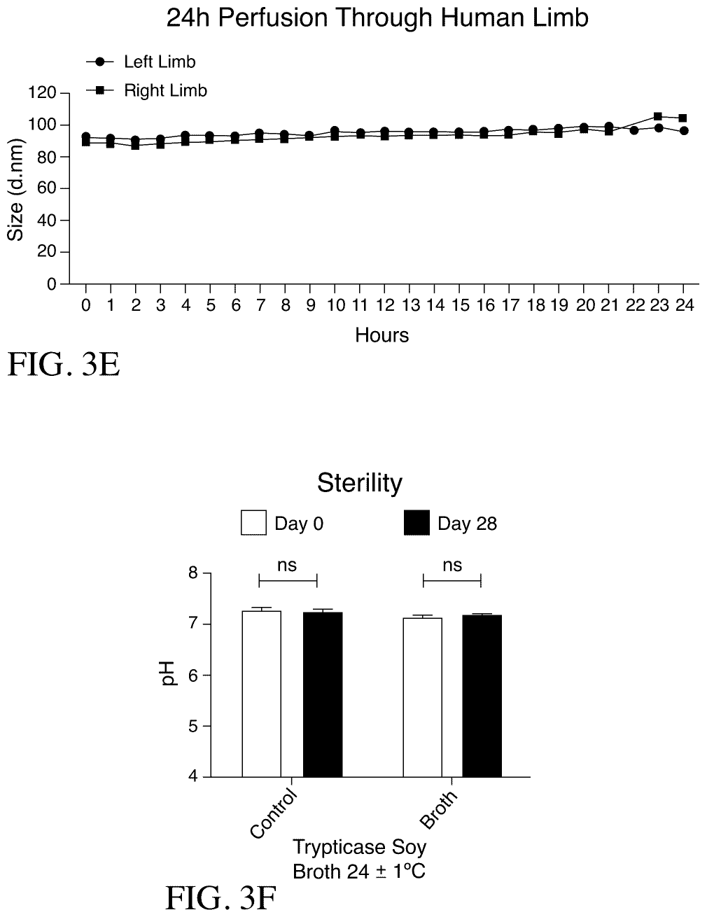

FIG. 2A shows the pre-mixing step of the manufacturing sequence of three phases (PFCs, hydrocarbon oils, and buffered, isotonic aqueous phase), which is followed by microfluidization on a microfluidizer processor and the final bulk product divided into 2 bottles for storage. Scaled-up fluorocarbon nanoemulsion formulations had their colloidal and fluorescence properties remained unchanged (FIGS. 2B-2C) and showed exceptional batch-to-batch reproducibility (FIG. 2D). The large-scale stabilized fluorocarbon nanoemulsion formulations have small droplet size (90-110 nm), incorporate clinical grade human use approved near-infrared fluorescent (NIRF) dye, narrow size distribution (<0.12), FIG. 2B, optimal pH, oncotic and osmotic pressure, and exceptionally stable under stress and during machine perfusion conditions (FIGS. 3A-3F). The large-scale stabilized fluorocarbon nanoemulsion formulations are exceptionally stable upon storage with no change in droplet size distribution over 300 days (FIG. 3A) or fluorescence signal after 12 months (FIG. 3B). In a representative experiment, large-scale stabilized fluorocarbon nanoemulsion formulations were run through a pair of human limbs for 24 h on a closed-circuit using BMI OrganBank™ VCA Preservation Machine. During the perfusion, the large-scale stabilized fluorocarbon nanoemulsion formulations were subjected to the effects of the centrifugal pump, passing through a membrane-based oxygenator before reaching the human limbs. Remarkably the large-scale stabilized fluorocarbon nanoemulsion formulations remained colloidally stable without any change in size distribution as monitored by Dynamic Light Scattering every hour throughout the experiment (FIGS. 3C, 3E).

For the reliable NIRF monitoring of the large-scale stabilized fluorocarbon nanoemulsion formulation perfusion through the system and the limbs, they also must maintain the fluorescence signal unchanged (FIG. 3D). Furthermore, the PFC-NEs pass the 28-days sterility testing requirement per the USP guidelines (FIG. 3F). As they are generated from GRAS materials, the large-scale stabilized fluorocarbon nanoemulsion formulations are non-toxic in vitro or in vivo.

As shown in Table 1, the large-scale stabilized fluorocarbon nanoemulsion formulations had an unexpected combination of desirable properties including small droplet size, narrow size distribution, safe components, demonstrated oxygen loading/release, and exceptional stability during circulation in machine perfusion systems.

TABLE 1

| Critical Quality Attribute (CQAs) | Target / specification | Assessment |
|---|---|---|
| Droplet size (z-average) and | Size + 100 ± 10 nm | Dynamic Light |
| size distribution | Dispersity ≤0.2 | Scattering (DLS) on |
| Zeta potential (ZP) | −15 ± 10 mV | Zeta Sizer Nano ZS, |
| Colloidal Stability (Droplet size | ≤20% size change upon | Phase Contrast |
| change upon storage) | storage for 12 months | Microscopy |
| Size distribution stability | Dispersity ≤0.2 at 12 months | |
| Centrifugation, Filtration, | ≤20% diameter change, | |
| Exposure to biological media | Dispersity ≤0.2 | |
| Machine perfusion and | ≤20% diameter change | |
| oxygenation | Dispersity ≤0.2 | |
| Machine Perfusion Conditions | ≤20% diameter change | |
| with/without tissues | Dispersity ≤0.2 | |
| PFC content | 30 ± 2% | 19F NMR |
| Near-infrared fluorescence | ≤10% change upon storage | LiCOR Odyssey |
| (NIRF) signal stability | 12 months | Imager, Microplate |
| NIRF change upon stress: | ≤10% change | reader |
| filtration, machine perfusion | | |
| condition, exposure to biomedia | | |
| Oxygenation capacity | Full saturation after <10 min | PreSense |
| | of oxygenation with a | Fiber-Optic Oxygen |
| | membrane oxygenator | Microsensor |
| Rheological Behavior | Low Viscosity less than or | Discovery Hybrid |
| | equal to plasma | Rheometer HR 20 |
| pH, oncotic and osmotic pressure | Physiological ± 10% | pH meter, osmometer |
| Cell viability | 90 ± 10% upon 24 h | Cell Titer Glo, Cell |
| | cell exposure | Titer Green (Promega) |

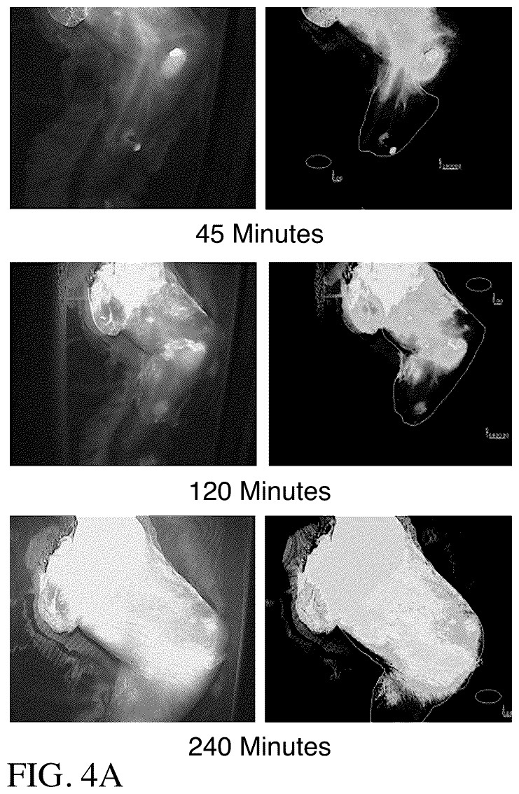

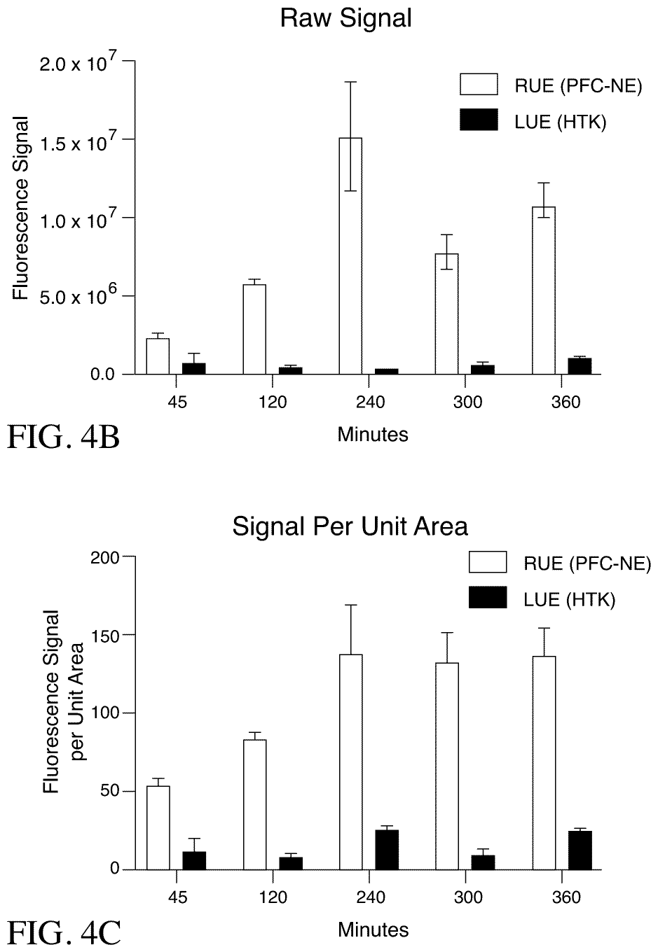

In addition, the large-scale stabilized fluorocarbon nanoemulsion formulations may be labeled with NIRF dyes for the purpose of in vivo and ex vivo detection of the perfusing nanoemulsion droplets in organs, tissues, and cells. The NIRF dyes may be stably incorporated into the large-scale stabilized fluorocarbon nanoemulsion formulations droplets during manufacturing and does not change in response to oxygenation of the large-scale stabilized fluorocarbon nanoemulsion formulations are shown in FIG. 3D. Stable NIRF labeling of large-scale stabilized fluorocarbon nanoemulsion formulations allows for continuous monitoring of their passage through tissues in limbs, both human and non-human, solid organs and biomedically engineered organs during machine perfusion. FIGS. 4A-4C show that NIRF signal from large-scale stabilized fluorocarbon nanoemulsion formulations circulated through non-human primate limbs over time as the machine perfusion continues. The NIRF image processing and signal quantification methodology may further be adapted to human limb perfusion monitoring, human solid organs, tissues or biomedically engineered organs, or xenografts etc. The methods described here for monitoring by NIRF imaging perfusion are applicable and not limited to nanoemulsions, microemulsions, micelle, proteinaceous solutions, colloidal solutions, or any type of organ/tissue preservation solution that carries ICG or any other NIRF dye for the means of monitoring perfusion fluid through any tissue or organ including limbs, eyes, faces.

The large-scale stabilized fluorocarbon nanoemulsion formulations may be produced by methods including a microfluidization step using a microfluidizer at pressures of from 15,000 psi to about 18,000 psi. The large-scale stabilized fluorocarbon nanoemulsion formulations droplet sizes are uniform with a low polydispersity index.

The large-scale stabilized fluorocarbon nanoemulsion formulations may include hydrocarbon lipids, fluorocarbons, water, nonionic surfactants, optional quaternary ammonium compounds.

The large-scale stabilized fluorocarbon nanoemulsion formulations may include a dye or NIRF dye, such as a tricarbocyanine dye, such as indocyanine green (ICG).

The large scale stabilized fluorocarbon nanoemulsion formulations may be produced such that the nanoemulsion formulations have long-term colloidal stability.

The scale stabilized fluorocarbon nanoemulsion formulations may be produced such that droplets of the nanoemulsion have a droplet size of from about 90 nm to about 120 nm, of from about 95 nm to about 115 nm, or from about 100 nm to about 110 nm, and wherein the diameter of the droplets does not change by more than 20%, or more than 15% or more than 10% or more than 5%, upon storage for at least 12 months.

The scale stabilized fluorocarbon nanoemulsion formulations may be produced such that droplets of the nanoemulsion formulations have a polydispersity index of less than about 0.2 and wherein the polydispersity index is less than about 0.2 after the nanoemulsion has been stored for at least 12 months.

The scale stabilized fluorocarbon nanoemulsion formulations may be produced such that the diameter of the droplets does not change by more than 20% or more than 15% or more than 10% or more than 5% upon centrifugation, oxygenation, filtration or exposure to biological media.

The large-scale stabilized fluorocarbon nanoemulsion formulations may include hydrocarbons, fluorocarbons, water, surfactants such as Pluronic P105, F127, P123, and/or Cremophor EL, dyes such as indocyanine green, quaternary ammonium compounds such as octadecylamine, and co-solubilizers such as 2-(2-ethoxyethox)ethanol or transcutol.

Hydrocarbons used in the preparation of the large-scale stabilized nanoemulsion formulations may include lipids such as mono-, di- and/or triglycerides with C8- to C22-fatty acids, such as C8- to C18-fatty acids, and/or fat-soluble vitamins. Examples of hydrocarbons may also include lipids or natural oils, such as, for example, groundnut oil, almond oil, olive oil, sesame oil, soybean oil, thistle oil (safflower oil) or cotton oil, semisynthetic oils, such as, for example, medium-chain triglycerides (MCTs), a triglyceride mixture which comprises principally C8- to C12-fatty acids, in particular caprylic acid and capric acid, as fatty acids, but also the fat-soluble vitamins (vitamin A, vitamin D, vitamin E, vitamin K).

Fluorocarbons used in the preparation of the large-scale stabilized nanoemulsion formulations may include perfluorocarbons, such as perfluorooctylbromide, perfluoropolyethers, perfluoro-15-crown-5 ether, perfluorodecaline, and/or perfluorooctane.

In some aspects, the quaternary ammonium compound and dye may be in the form of zwitterions.

The large-scale stabilized nanoemulsion formulations also showed excellent colloidal stability under stress from centrifugation and when exposed to biological medium. All large-scale stabilized nanoemulsion formulations displayed a narrow size distribution with a PDI of less than 0.15.

The method of manufacturing large-scale stabilized nanoemulsion formulations may include the preparation of a pre-mixed solution including a hydrocarbon, a co-solubilizer and a dye solution with a quaternary ammonium compound. The method may further include the addition of a fluorocarbon, such as a perfluorocarbon, and a surfactant aqueous solution to the pre-mix to form a pre-emulsion solution. The method may further include the mixing and blending of the pre-emulsion solution to form a crude emulsion. The method may further include the emulsification of the crude emulsion via multiple passages through a microfluidizer. The method may further include the addition of a surfactant aqueous solution to the crude emulsion after passage through the microfluidizer.

### Methods of Manufacturing Scalable Stabilized Nanoemulsion Formulations

The method of manufacturing the large-scale scalable stabilized nanoemulsion formulations may include pre-mixing a solution including a hydrocarbon, a co-solubilizer and a dye solution comprising a quaternary amine to form a pre-mix; adding a fluorocarbon and a surfactant aqueous solution to the pre-mix to form a pre-emulsion solution; mixing and blending of the pre-emulsion solution to form a crude emulsion; adding a surfactant aqueous solution to the crude emulsion; and emulsifying the crude emulsion via multiple passages through a microfluidizer.

The method of manufacturing the large-scale scalable stabilized nanoemulsion formulations may be such that when the dye solution is incorporated into the scalable stabilized nanoemulsion, the diameter of the droplets does not change by more than 20%.

### Methods of NIRF Imaging and Perfusate Detection During Machine Perfusion

The method for quantitative and qualitative monitoring of perfusates through limbs (FIG. 4) relies on stable incorporation of NIRF dye into fluorocarbon nanoemulsion that is circulated through by machine perfusion. The NIRF monitoring includes both live/continuous monitoring during the perfusion and post-perfusion assessment of the distribution of NIRF-labeled perfusion fluids by any means (direct conjugations, adsorption or protein binding) including NIRF-labeled fluorocarbon nanoemulsions, microemulsions, colloidal solutions, proteinaceous solutions, etc. The NIRF monitoring is performed by clinical or preclinical grade imagers research-grade or commercially available that are able to detect NIRF dye in tissues, organs and VCA. The NIRF monitoring includes collection of images, storage, processing and quantification of signal (FIG. 4). The NIRF monitoring includes correlating the detection of NIRF signal from perfusates being circulated through VCA, organs or tissues by any type of perfusion machine available on the market, or research-grade machine. The methods presented here are applicable to any machine perfusion and any type of NIRF detection of dyes such as indocyanine green or other type of NIRF dyes included by any means in any perfusate running through VCA, organs or tissues for the means of monitoring perfusion efficiency in real time or sequentially, during perfusion or for the purpose of post assessments.

NIRF imaging method is adaptable to any perfusate carrying dyes such as indocyanine green or any other NIRF dye. NIRF imaging method shown here is applicable to imaging and/or detection of ICG perfused by machine perfusion through limbs, faces, whole eye, penis, abdominal wall, flaps etc (collectively known as VCA), solid organs (e.g. heart, lungs, kidneys), and tissues.

As shown in FIGS. 4A-4C, the NIRF imaging method is adaptable to any perfusate carrying dyes such as indocyanine green or any other NIRF dye. NIRF imaging method shown in FIGS. 4A-4C is applicable to imaging and/or detection of dyes such as indocyanine green perfused by machine perfusion through limbs, faces, whole eye, penis, abdominal wall, flaps etc (collectively known as VCA), solid organs (e.g. heart, lungs, kidneys), and tissues.

Dyes or NIRF dyes such as indocyanine green may be stabilized by being incorporated into buffers, vital solutions, protein containing solutions, commercial and other organ preservation solutions as a nanoemulsion, micelle, lipid or surfactant conjugate to assure stable labeling of the perfusate for the purpose of monitoring by NIRF detection and/or imaging of the machine perfusion of organs, limbs, tissues, etc.

Suitable buffers may include, for example phosphate buffer solution (PBS), borate buffer, triethanolamine/minodiacetic acid, tris (2-amino-2-hydroxymethyl-propane-1,3-diol), sodium phosphate, ammonium acetate, magnesium acetate tetrahydrate, sodium acetate, phosphate citrate, tricine (N-(Tri(hydroxymethyl)methyl)glycine), bicine (2-(bis(2-hydroxyethyl)amino)ethanoic acid), EDTA, TES, HEPES, TAPS (N-Tris(hydroxymethyl)methyl-3-aminopropanesulfonic acid), glycine, glycylglycine, imidazole, MES, MOPS, PIPES, potassium phosphate, sodium phosphate, tricine, triethanolamine, tris base, trisodium citrate, Krebs-Ringer HEPES buffer, or any commonly used biological buffers and buffer constituents.

The methods according to the invention may include labeling any perfusate with dyes such as indocyanine green stably for the purpose of monitoring perfusion by NIRF imaging and/or detection.

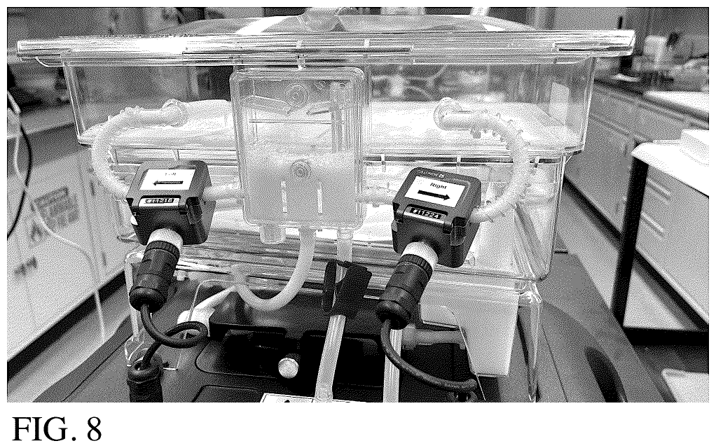

As seen in FIG. 8, the NIRF dye labeled nanoemulsions according to the invention may be run in any non-commercial or commercial grade perfusion systems.

### EXAMPLES

### Example 1. Preparation of Surfactant Solution

A solution of 168 mL 10× phosphate buffered saline (PBS) and 832 mL HPLC water was stirred for at least 20 minutes at 250 RPM. 20 g Pluronic P105 and 30 g Cremophor EL (CrEL) were separately added, combined, and stirred into the previous solution to form a surfactant solution.

### Example 2. Preparation of Dye Solution

A solution of indocyanine green-octadecylamine in 1:1.8 molar ratio was prepared from 100 mg indocyanine green and 62.52 mg octadecylamine in 600 mL DMSO:transcutol solvent (1:1 v/v).

### Example 3. Preparation of Nanoemulsion Formulation 600 mL

36 g Miglyol 812N, 6.5 g transcutol, and 0.75 mL dye solution were pre-mixed at room temperature at 250 RPM for several hours. 72 g perfluorooctylbromide and about 100 g of the surfactant solution of Example 1 were added to the pre-mix and stirred at 700 RPM for at least 15 minutes. The resulting pre-emulsion was blended during a few seconds yielding a crude emulsion. A portion of surfactant solution was passed trough the line of a microfluidizer. The crude emulsion was then passed through the microfluidizer set at a pressure of 18,000 psi. The resulting emulsion was collected and was passed through the microfluidizer 3 times after successful additions totaling about 450 g of surfactant solution and blending resulting in a 600 mL stabilized nanoemulsion formulation.

### Example 4. Preparation of Nanoemulsion Formulation 1.2 L

72 g Miglyol 812N, 13 g transcutol, and 1.5 mL dye solution were pre-mixed at room temperature at 250 RPM for several hours. 360 g perfluorooctylbromide and about 190 g of the surfactant solution of Example 1 were added to the pre-mix and stirred at 700 RPM for at least 15 minutes. The resulting pre-emulsion was blended during a few seconds yielding a crude emulsion. A portion of surfactant solution was passed through the line of a microfluidizer. The crude emulsion was then passed through the microfluidizer set at a pressure of 18,000 psi. The resulting emulsion was collected and was passed through the microfluidizer 3 times after successful additions totaling about 640 g of surfactant solution and blending resulting in a 1.2 L stabilized nanoemulsion formulation.

### Example 5. Preparation of Nanoemulsion Formulation 2.4 L

144 g Miglyol 812N, 25.3 g transcutol, and 3 mL dye solution were pre-mixed at room temperature at 250 RPM for several hours. 720 g perfluorooctylbromide and about 480 g of the surfactant solution of Example 1 were added to the pre-mix and stirred at 700 RPM for at least 90 minutes. The resulting pre-emulsion was blended during a few seconds yielding a crude emulsion. A portion of surfactant solution was passed through the line of a microfluidizer. The crude emulsion was then passed through the microfluidizer set at a pressure of 18,000 psi. The resulting emulsion was collected and was passed through the microfluidizer 3 times after successful additions totaling about 1,857 g of surfactant solution and blending resulting in a 2.4 L stabilized nanoemulsion formulation.

### Example 6. Limb Preservation Trials

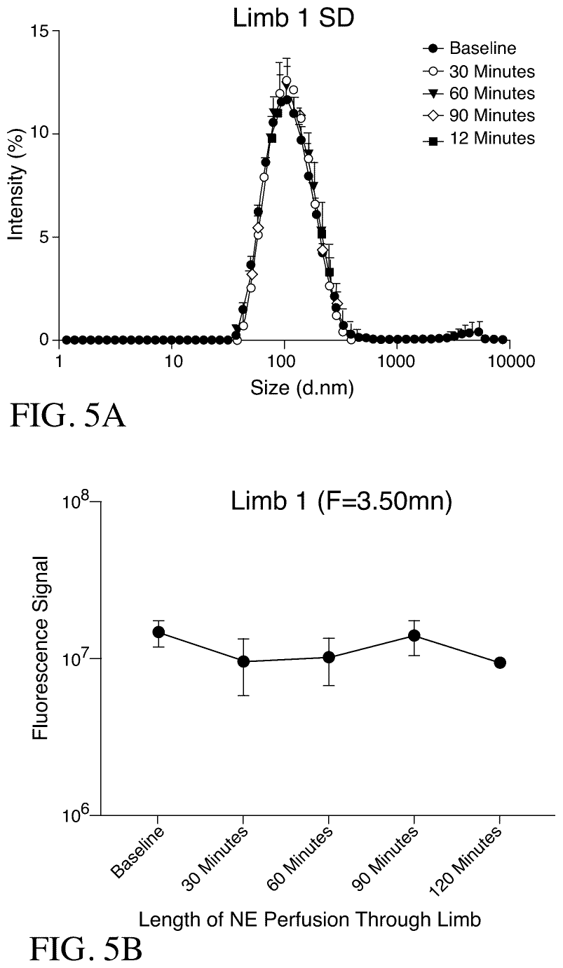

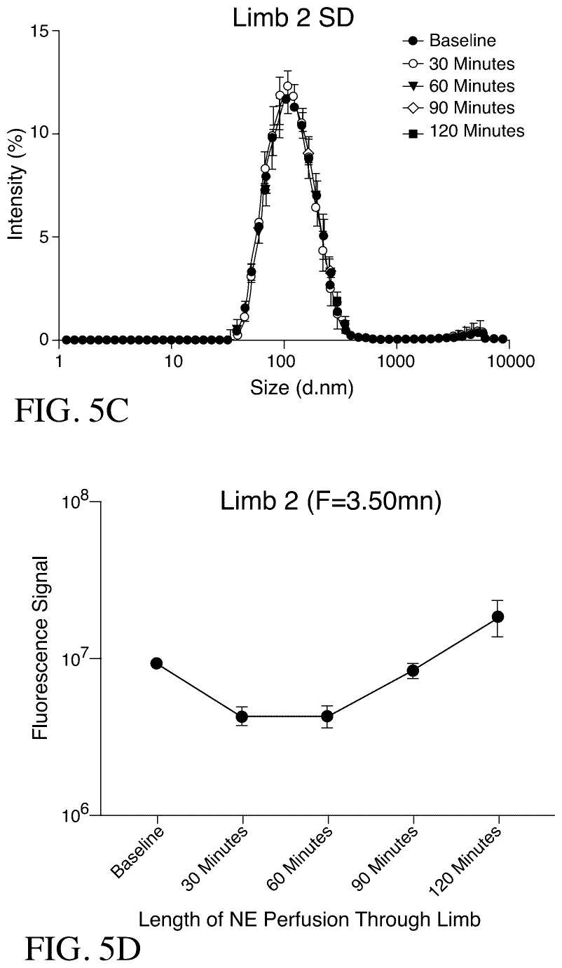

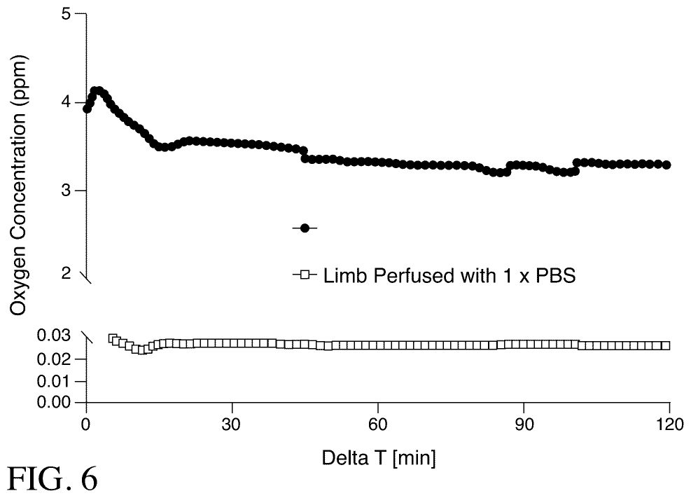

To mimic the successfully completed human limb preservation trials, sample collection of the perfused nanoemulsion formulation of Example 5 was performed to assess size and fluorescence on the product post-perfusion. Samples were collected every 30 minutes and compared to a baseline measurement of just oxygenated nanoemulsion formulation before exposure to rat hindlimb tissue. Results are shown in FIGS. 5A-5D. As shown, the size of the perfused nanoemulsion formulation and fluorescence signal is consistent throughout the full two-hour perfusion experiment. This mirrors results collected from the human limb preservation experiment and was expected. This data completes the claim that the nanoemulsion formulation will maintain its colloidal and fluorescence integrity after perfusion throughout the limb.

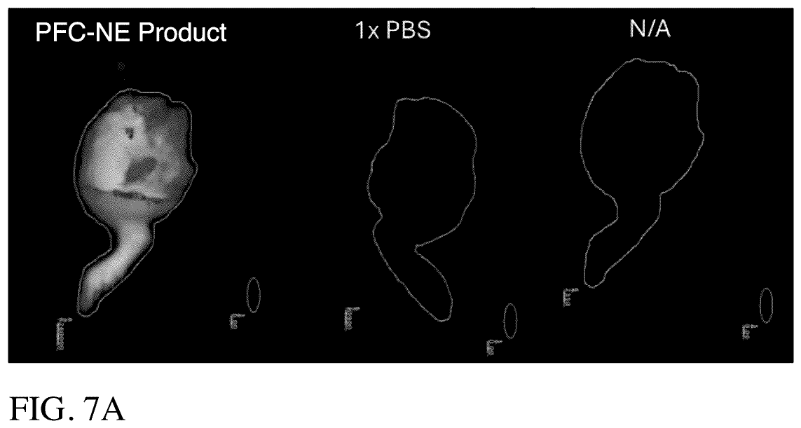

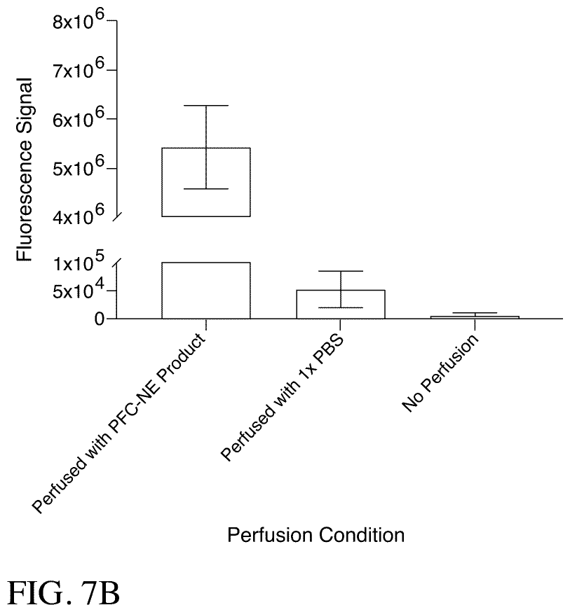

Furthermore, oxygen concentration was measured throughout the two-hour perfusion experiment and compared between limbs perfused with nanoemulsion formulation and the limb perfused with just 1× phosphate buffered saline (PBS). A significant difference was seen in the measured oxygen in the skeletal tissue of the rat's hindlimb between the two conditions, with the rat limb receiving nanoemulsion formulation measuring to approximately 3 ppm and the rat limb receiving 1×PBS measuring to roughly 100× less oxygenation at approximately 0.03 ppm. This data can be visualized in FIGS. 7A-7B.

FIGS. 7A-7B show NIRF imaging that was performed on the limbs to confirm fluorescence of the large-scale nanoemulsion formulations within the limb. Ex vivo imaging performed upon completion of the study on the limb perfused with PFC-NE product, the limb perfused with 1×PBS, and the limb stored on ice for two hours. As expected, fluorescence signal is two orders of magnitude higher in the limb perfused with large-scale stabilized fluorocarbon nanoemulsion formulations than the limb perfused with 1×PBS. Negligible signal was measured in the limb preserved on ice. Rat cannulation studies were successfully performed and designed to mimic previously reported human limb preservation trials.

We claim:

1. A formulation for oxygen delivery for organ preservation comprising a scalable stabilized nanoemulsion, the nanoemulsion comprising:
(a) a hydrocarbon lipid;
(b) a fluorocarbon or perfluorocarbon;
(c) water,
(d) a nonionic surfactant; and
(e) optionally quaternary ammonium compound,
wherein droplets of the nanoemulsion have a droplet size of from about 90 nm to about 120 nm and wherein the diameter of the droplets does not change by more than 20% upon storage for at least 12 months.

2. The formulation of claim 1, wherein droplets of the nanoemulsion have a polydispersity index of less than about 0.2 and wherein the polydispersity index is less than about 0.2 after the nanoemulsion has been stored for at least 12 months.

3. The formulation of claim 1, wherein the diameter of the droplets does not change by more than 20% upon centrifugation, filtration or exposure to biological media.

4. The formulation of claim 1, wherein the diameter of the droplets does not change by more than 20% upon oxygenation of the formulation.

5. The formulation of claim 1 further comprising a buffer solution.

6. The formulation of claim 1 further comprising a tricarbocyanine dye.

7. The formulation of claim 1, wherein the fluorocarbon is a perfluorocarbon.

8. The formulation of claim 1, wherein the quaternary ammonium compound comprises octadecylamine.

9. A method of preparing the scalable stabilized nanoemulsion of claim 1 comprising
(a) pre-mixing a solution including a hydrocarbon, a co-solubilizer and a dye solution comprising a quaternary amine to form a pre-mix;
(b) adding a fluorocarbon and a surfactant aqueous solution to the pre-mix to form a pre-emulsion solution;
(c) mixing and blending of the pre-emulsion solution to form a crude emulsion;
(d) adding a surfactant aqueous solution to the crude emulsion; and
(e) emulsifying the crude emulsion via multiple passages through a microfluidizer.

10. The method of claim 8, wherein when the dye solution is incorporated into the scalable stabilized nanoemulsion, the diameter of the droplets does not change by more than 20%.

11. A method of using the scalable stabilized fluorocarbon nanoemulsion of claim 1 comprising administering the nanoemulsion via perfusion through limbs, wherein the limbs are preserved during Vascularized Composite Allotransplantation (VCA) and/or solid organ preservation.

12. A method for NIRF detection and monitoring of perfusion of preservation fluids, the method comprising administering to a tissue and/or an organ of a subject the scalable stabilized nanoemulsion of claim 1, and detecting two or more signals emitted by the administered nanoemulsion.

13. The method of NIRF detection and monitoring of perfusion of preservation fluids of claim 12, further comprising incorporating a NIRF dye during organ or tissue machine perfusion for the purpose of preservation prior to transplantation.

14. The method of NIRF detection and monitoring of perfusion of preservation fluids of claim 12, wherein the detecting step comprises the detection of signals collected from commercial NIRF signal detectors.

15. The method of NIRF detection and monitoring of perfusion of preservation fluids of claim 12, wherein the detecting step comprises the detection of signals collected from research-grade NIRF signal detectors.
